(19) United States
(12) Patent Application Publication
(10) Pub. No.: US 2018/0055685 A1
(43) Pub. Date: Mar. 1, 2018

(54) CRYOTHERAPY SYSTEM WITH INFLATABLE CHAMBER

(71) Applicant: Revo Cryotherapy, LLC, Davidson, NC (US)

(72) Inventor: Caio Campos, Davidson, NC (US)

(21) Appl. No.: 15/688,028

(22) Filed: Aug. 28, 2017

Related U.S. Application Data
(60) Provisional application No. 62/380,561, filed on Aug. 29, 2016.

(51) Int. Cl. A61F 7/00; A61H 33/06; A61H 33/00

(52) U.S. Cl. CPC A61F 7/0053; A61H 33/06; A61F 2007/0064; A61H 2033/062; A61H 2201/0214; A61H 33/6005

(57) ABSTRACT

A cryotherapy system can include an erectable apparatus that can be at least partially inflatable, and configured to extend at least partially around a treatment area adapted to contain at least both vaporized cryogen and at least a portion of a user's body to be cryogenically treated; and a cooling system configurable to provide vaporized cryogen to the treatment area.

### CROSS-REFERENCE TO RELATED APPLICATION

This application claims the benefit of U.S. Provisional Application No. 62/380,561 filed Aug. 29, 2016, which is incorporated herein by reference in its entirety.

### FIELD OF THE INVENTION

The present invention relates to cryotherapy and, more particularly, to a cryotherapy system that comprises a chamber.

### BACKGROUND

It is typical for known whole body cryotherapy systems to be relatively large and heavy, so that they are relatively difficult to move and install.

### SUMMARY

An aspect of this disclosure is the provision of a cryotherapy system comprising at least one inflatable chamber configured to extend at least partially around, and at least partially define, a treatment area configured to contain both vaporized cryogen and at least a portion of a user's body to be cryogenically treated; and a cooling system configurable to be in fluid communication with the treatment area and provide vaporized cryogen to the treatment area. A method can comprise inflating the at least one inflatable chamber, and operating the cooling system to provide vaporized cryogen to the treatment area while the at least one inflatable chamber is inflated.

The at least one inflatable chamber can define an access opening configured to provide ingress to and egress from the treatment area when the at least one inflatable chamber is inflated. The system can further comprise an apparatus configurable to at least partially close the access opening.

The at least one inflatable chamber can comprise fabric, define an interior space configured to contain air under pressure, and comprise a plurality of sewn seams configured to discharge air from the interior space into the treatment area to mix the vaporized cryogen.

In an aspect of this disclosure, the inflatable chamber can comprise at least one inflatable wall configured to extend at least partially around the treatment area at least when the at least one inflatable wall is inflated, an access opening configured to provide ingress to and egress from the treatment area at least when the at least one inflatable wall is inflated, and an apparatus configurable to at least partially close the access opening.

The at least one inflatable wall can face and at least partially define the treatment area, define an interior space configured to contain air under pressure, and can be configured to discharge air from the interior space into the treatment area to mix the vaporized cryogen.

The at least one inflatable wall can comprise a plurality of subchambers extending at least partially around the treatment area. The plurality of subchambers can comprise a plurality of inflatable subchambers extending at least partially around the treatment area.

The at least one inflatable wall can comprise a fabric inner sidewall facing and extending at least partially around the treatment area, and a fabric outer sidewall extending at least partially around the inner sidewall. The inner sidewall can comprise a plurality of fabric pieces respectively connected to one another by a plurality of sewn seams.

The cooling system can comprise at least one nozzle configured to spray cryogen into the treatment area so that the cryogen evaporates in the treatment area. The controlling system can further comprise a control system configured to control when the cryogen is sprayed into the treatment area via the at least one nozzle to control temperature within the treatment area.

Another aspect of this disclosure is the provision of an erectable apparatus configurable between an erected configuration and a collapsed configuration, wherein in the erected configuration the erectable apparatus is configured to define a treatment area configured to contain both vaporized cryogen and at least a portion of a user's body to be cryogenically treated; and a cooling system configurable to be in fluid communication with the treatment area and provide vaporized cryogen to the treatment area. The erectable apparatus can comprise at least one sidewall configured to extend at least partially around the treatment area when the apparatus is in the erected configuration, and a plurality of chambers configured to be inflated to at least partially support the at least one sidewall in the erected configuration.

In an example, the at least one sidewall can comprise an inner sidewall. The plurality of chambers can define an inflatable wall that includes the inner sidewall. The inflatable wall can further include an outer sidewall extending at least partially around the inner sidewall.

In another example, the at least one sidewall can be an inner sidewall. The erectable apparatus can further comprise an outer sidewall. A chamber of the plurality of cambers can comprise a portion of the inner sidewall and a portion of the outer sidewall, so that the chamber defines an interior space positioned between the portion of the inner sidewall and the portion of the outer sidewall. The interior space can be configured to contain air under pressure, and the inner sidewall can be configured to (e.g., can include sewn seams configured to) discharge air from the interior space into the treatment area to mix the vaporized cryogen.

A method can include inflating the chambers and operating the cooling system to provide vaporized cryogen to the treatment area.

The foregoing summary provides a few brief examples and is not exhaustive, and the present invention is not limited to the foregoing examples. The foregoing examples, as well as other examples, are further explained in the following detailed description with reference to accompanying drawings.

### BRIEF DESCRIPTION OF THE DRAWINGS

FIG. 1 is a front pictorial view of a cryotherapy system with its erectable apparatus or main chamber in an erected or inflated configuration, and a user substantially enclosed in a treatment area of the main chamber, in accordance with an embodiment of this disclosure.

FIG. 2 is a front pictorial view of the cryotherapy system of FIG. 1 with an open door, in accordance with an embodiment of this disclosure.

FIG. 3 is a rear pictorial view of the cryotherapy system of FIG. 1, in accordance with an embodiment of this disclosure.

FIG. 4 is a schematic, vertical cross-sectional view of the main chamber taken substantially along line 4-4 of FIG. 1, in accordance with an embodiment of this disclosure.

FIG. 5 is a schematic, horizontal cross-sectional view of the main chamber taken along line 5-5 of FIG. 4, in accordance with an embodiment of this disclosure.

FIG. 6 is an enlarged schematic view of a portion of FIG. 5.

FIG. 7 is a rear view of a control unit, wherein an access panel of the control unit has been removed to show interior components, in accordance with an embodiment of this disclosure.

FIG. 8 comprises a pictorial view of a nozzle assembly, in accordance with an embodiment of this disclosure.

FIG. 9 comprises a partially exploded view of a diffuser assembly, in accordance with an embodiment of this disclosure.

FIG. 10 is a partial view that depicts the diffuser assembly and a deflection panel mounted within treatment area of the main chamber, in accordance with an embodiment of this disclosure.

FIG. 11 is like FIG. 10 except, for example, that the diffuser assembly and deflection panel are shown exploded away from features to which they are removably mounted in FIG. 10, in accordance with an embodiment of this disclosure.

FIG. 12 is a pictorial view of the cryotherapy system with its erectable apparatus or main chamber in an at least partially unerected or at least partially uninflated configuration, in accordance with an embodiment of this disclosure.

### DETAILED DESCRIPTION

The present invention embraces a cryotherapy system comprising an erectable apparatus that can be at least partially inflatable, and configured to extend at least partially around a treatment area adapted to contain at least both vaporized cryogen and at least a portion of a user's body to be cryogenically treated; and a cooling system configurable to provide vaporized cryogen to the treatment area. The erectable apparatus can comprise at least one inflatable chamber. The at least one inflatable chamber can be configured to extend at least partially around, and at least partially define, the treatment area. Examples of one or more embodiments of a cryotherapy system are described in the following.

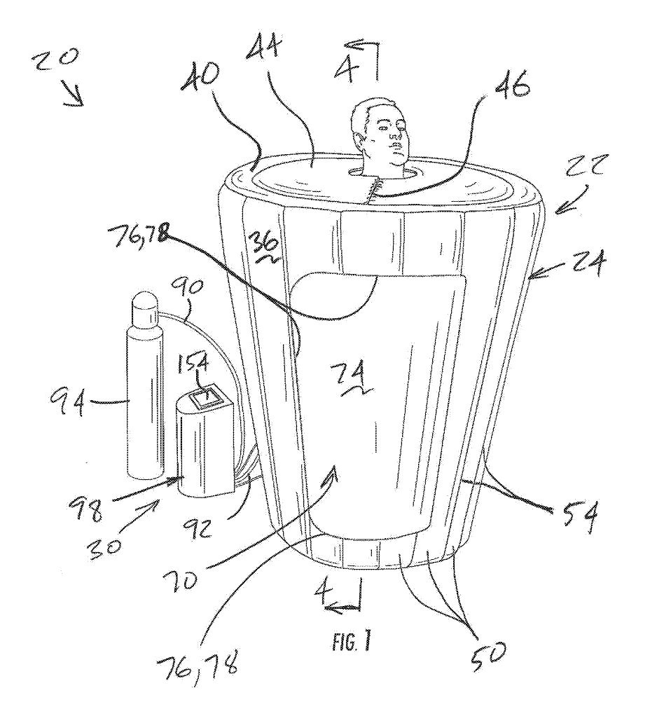

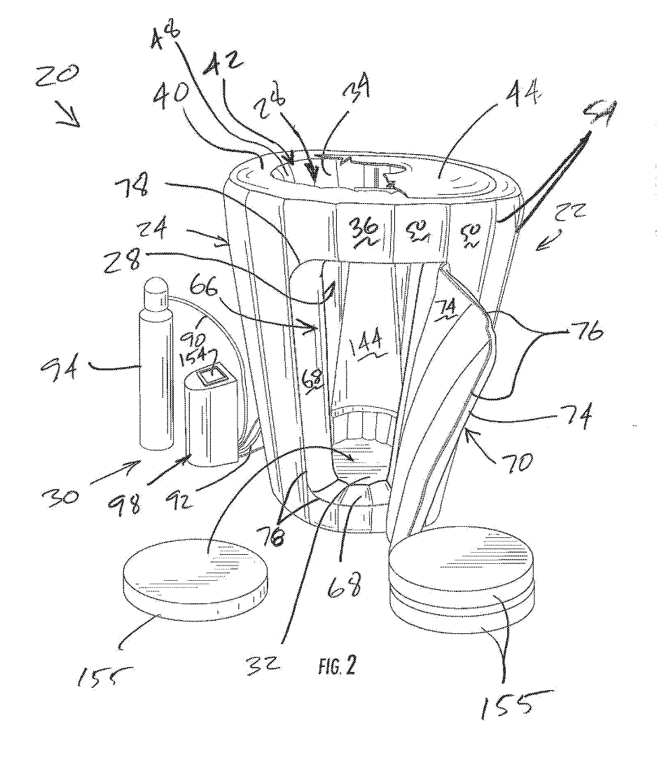

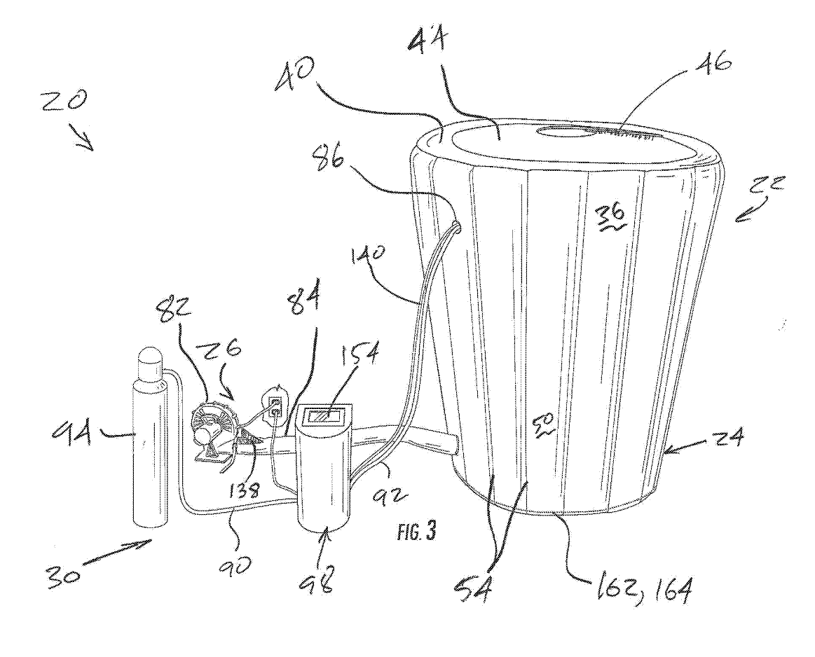

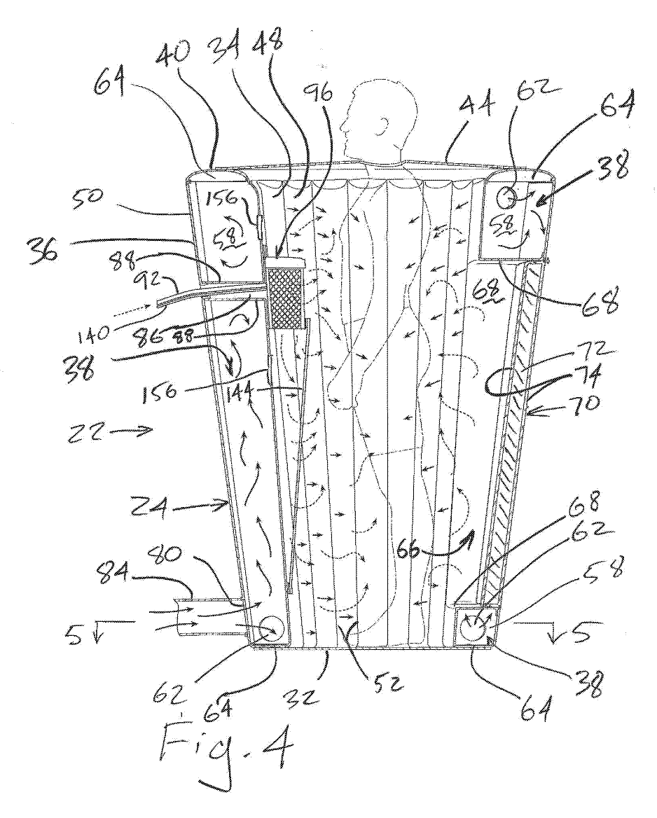

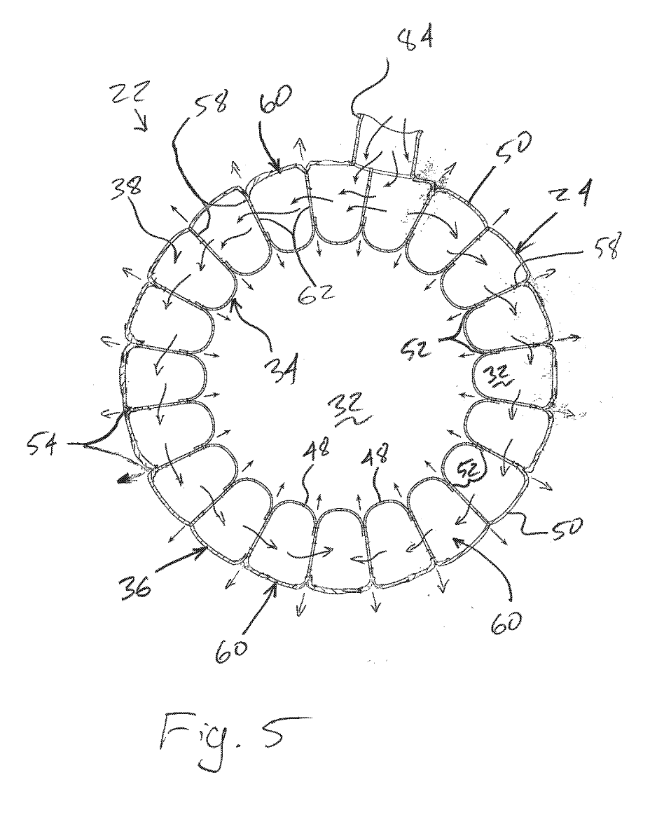

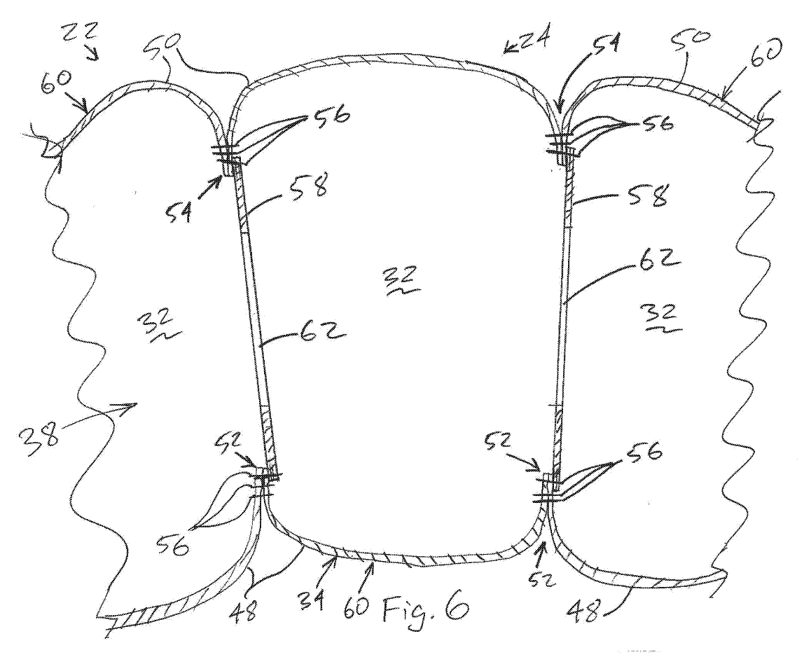

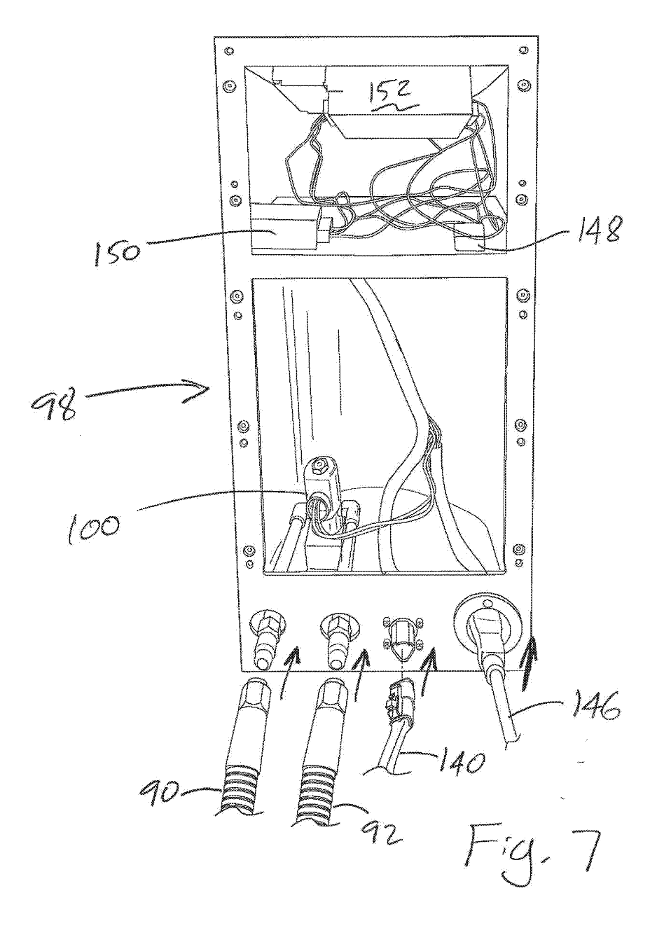

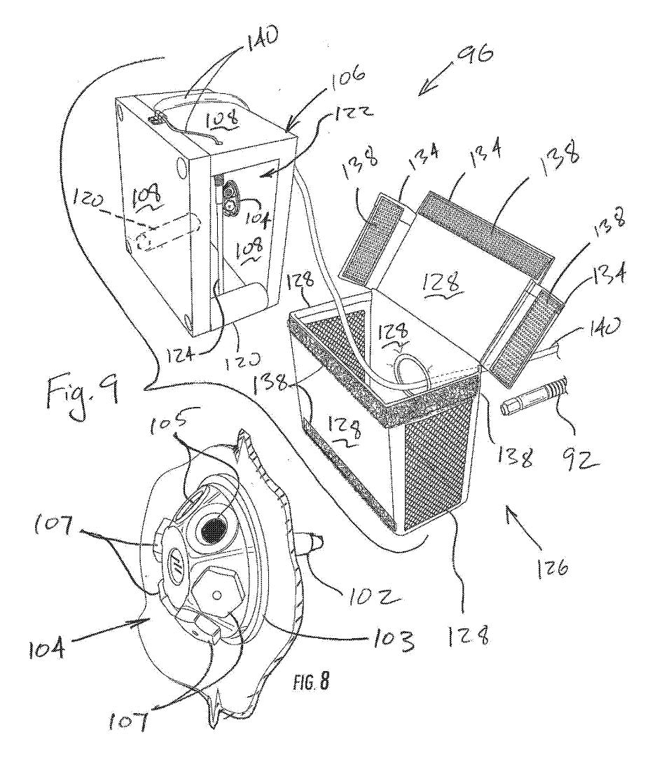

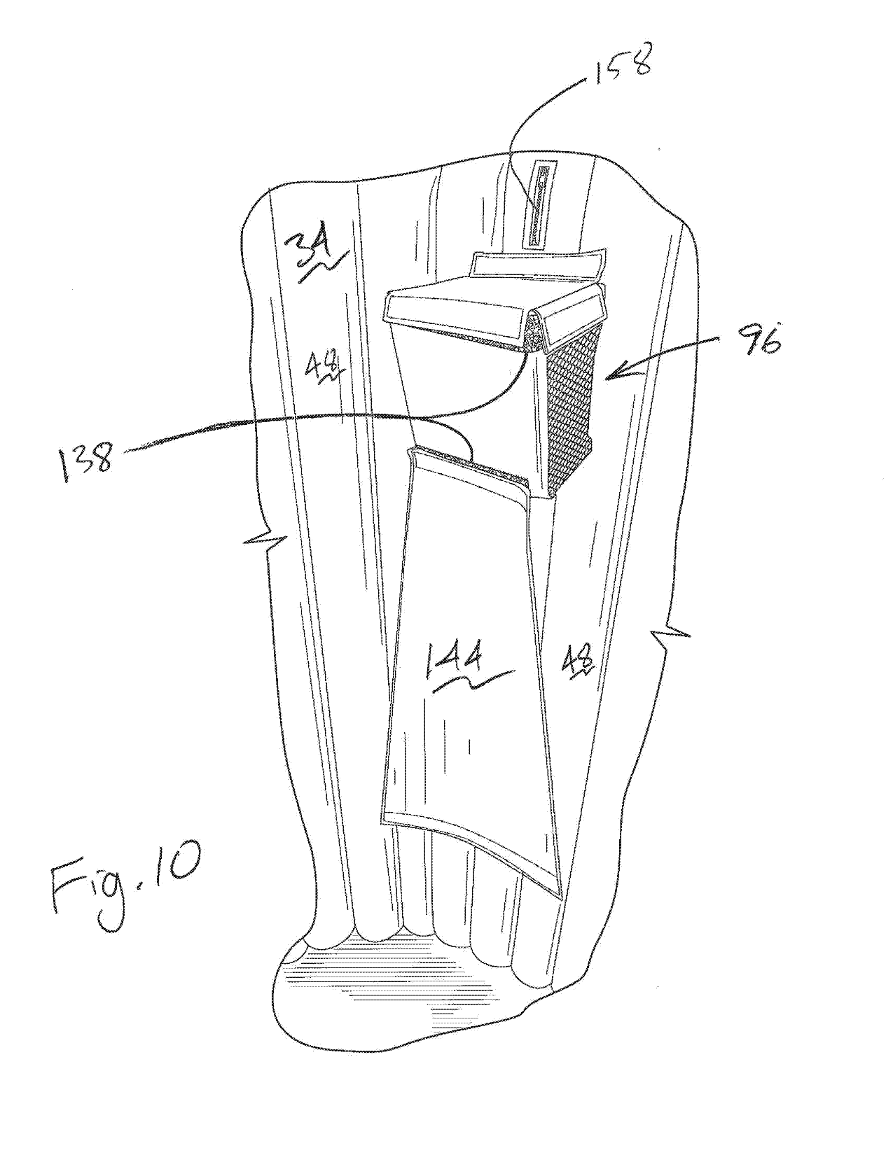

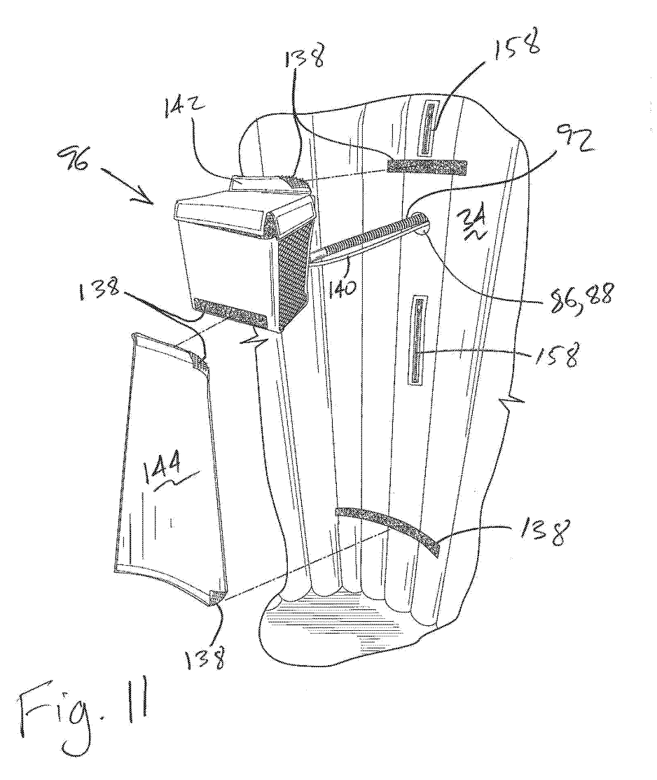

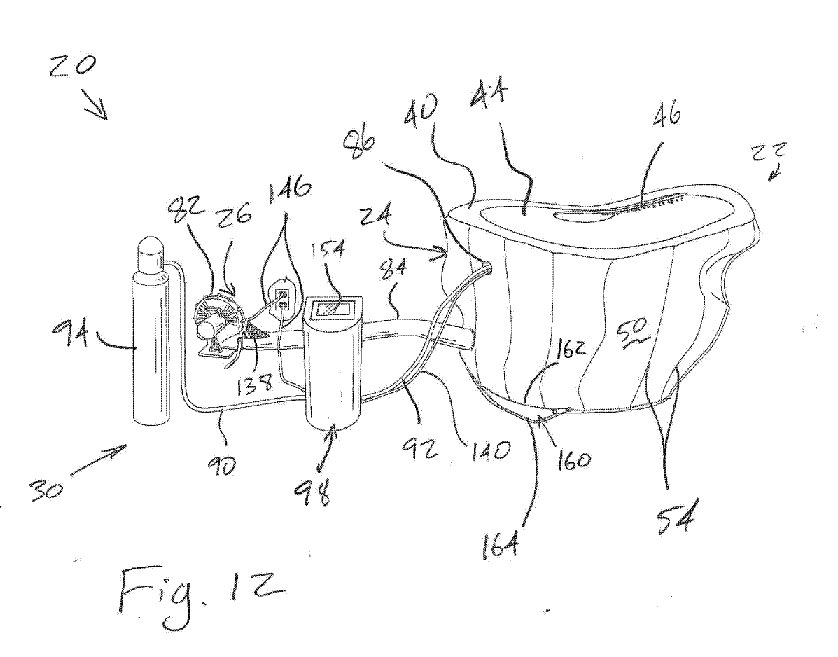

FIGS. 1-3 depict features of a cryotherapy system 20 of an embodiment of this disclosure. The cryotherapy system 20 includes an erectable apparatus or main chamber 22 that is depicted in an erected configuration in FIGS. 1-3. In the embodiment depicted in the drawings, the main chamber 22 comprises at least one inflatable wall 24, and the cryotherapy system 20 further includes an inflation system 26 (FIG. 3) for inflating the inflatable wall (e.g., with ambient air, or the like) and, thus, causing the main chamber to be in its erected configuration. The erected main chamber 22 defines an interior treatment area 28 (FIG. 2) configured to contain at least both vaporized cryogen (e.g., vaporized liquid nitrogen) and at least a portion of a user's body to be cryogenically treated. For example, FIG. 1 depicts a user substantially enclosed in the treatment area 28 of the main chamber 22. The cryotherapy system 20 typically further includes a cooling system 30 for selectively providing the vaporized cryogen to the treatment area 28, as will be discussed in greater detail below.

Referring to FIG. 2, the main chamber 22 can include a disk-shaped, fabric base panel 32, and the inflatable wall 24 can extend upwardly and outwardly from a periphery of the base panel, so that (e.g., in side elevation views) the inflatable wall, or more generally the main chamber, can be tapered or substantially frustoconical in its inflated configuration. The base panel 32 can be an uninflatable structure that closes the lower end of the treatment area 28, although variations are within the scope of this disclosure.

Referring to the schematic cross-sectional view of FIG. 4, the inflatable wall 24 can include fabric inner and outer sidewalls 34, 36 between which can be defined an interior space 38 of the inflatable wall. In the embodiment depicted in the drawings, the inner sidewall 34 faces, at least partially defines, and extends at least partially around the treatment area 28. More specifically regarding the embodiment depicted in the drawings, the inner sidewall 34 partially defines, and extends completely around, the treatment area 28.

In the embodiment depicted in the drawings, the interior space 38 of the inflatable wall 24 (“wall interior space”) is substantially sealed closed for receiving air under pressure from the inflation system 26, for inflating the inflatable wall. For example, for substantially closing the lower and upper ends of the wall interior space 38, lower annular edges of the inner and outer sidewalls 34, 36 can be securely connected to the base panel 32 by respective sewn seams, and upper annular edges of the inner and outer sidewalls can be securely connected to an annular fabric upper panel 40 of the main chamber 22 by respective sewn seams. More specifically, the lower edge of the outer sidewall 36 can be connected to the outer peripheral edge or margin of the base panel 32, and the lower edge of the inner sidewall 34 can be connected to a more centrally located portion of the base panel. Similarly, the upper edge of the outer sidewall 36 can be connected to an outer peripheral edge or margin of the upper panel 40, and the upper edge of the inner sidewall 34 can be connected to an inner peripheral edge or margin of the upper panel. The upper panel 40 is typically not inflatable, although variations are within the scope of this disclosure.

The upper panel 40 can be centrally open, so that the upper panel and optionally also the upper edge of the inner sidewall 34 extend around and define an upper opening 42 (FIG. 2) to the treatment area 28. More specifically, in the embodiment depicted in the drawings, the main chamber 22 is configured so that the treatment area 28 is at least partially upwardly open, such as for having the neck and head of a user extend outwardly therefrom. Accordingly, the cryotherapy system 20 may be referred to as a partial body cryotherapy system, although it may also be referred to as a whole body cryotherapy system.

The upper panel 40 and/or another suitable structure (e.g., a fabric connected by a sewn seam to the upper panel) can extend inwardly past the inner sidewall 34 to form an adjustable, flexible collar or flange 44 for selectively at least partially obstructing the upper opening 42 to the treatment area 28. The flange 44 can be configured to extend around the neck of a user standing in the treatment area 28, so that the flange at least partially closes or obstructs the upper opening 42 to the treatment area. A slit or other suitable cut can extend outwardly from a central opening of the flange 44 for adjustability, so that the opposite edges of the flange formed by the slit can be selectively joined together by a releasable fastener that can be in the form of a zipper 46 that is schematically depicted in FIGS. 1 and 3. The opposite interlinkable strips of the zipper 46 can be respectively sewn to the edges of the flange 44 that are formed by the slit in the flange.

As best understood with reference to the schematic cross-sectional views of FIGS. 4-6, the inner and outer sidewalls 34, 36 can respectively include a series of fabric pieces or inner and outer wall strips 48, 50 having opposite ends respectively connected to the base and upper panels 32, 40 by the respective sewn seams mentioned above. The edges of adjacent inner strips 48 can be securely connected to one another by inner sewn seams 52 extending from the top to the bottom of the main chamber 22. The edges of adjacent outer strips 50 can be securely connected to one another by outer sewn seams 54 extending from the top to the bottom of the main chamber 22. Referring to the horizontal cross-sectional view of FIG. 6, the sewn seams 52, 54 can comprise upright marginal portions of adjacent wall strips 48, 50 being in opposing face-to-face contact with one another and substantially fixedly joined together by one or more sewn stiches 56 extending along the entire length of the wall strips 48, 50.

The inflatable wall 24 can further include a series of fabric pieces or partition strips 58 spanning across the wall interior space 38. Referring to FIG. 6, upright marginal portions of the partition strips 58 can be in opposing face-to-face contact with respective upright marginal portions of the inner and outer strips 48, 50 and fixedly joined thereto by sewn stiches 56 extending along the entire lengths of the partition strips. Accordingly, the inflatable wall 24 can comprise a series subchambers 60 extending at least partially around, or completely around the treatment area 28. Adjacent subchambers 60 may be in fluid communication with one another by way of one or more distribution holes 62 in the partition strips 58 and/or by virtue of gaps 64 (FIG. 4) between upper and lower ends of the partitions strips and the base and upper panels 32, 40, or the like. The main chamber 22 and/or inflatable wall 24 may be referred to as substantially frustoconical in its inflated configuration because, for example, the partition strips 58 can at least partially restrict relative movement between the inner and outer sidewalls 34, 36 in a manner that causes the inner and outer sidewalls to at least partially have or define wavy or undulating shapes.

There can be one or more openings from outside of the main chamber 22 to the treatment area 28 defined by the main chamber, and such openings can extend through the inflatable wall 24 or be formed in any other suitable manner. In the embodiment depicted in FIG. 2, a main access opening 66 extends through the inflatable wall 24 for providing a user ingress to, and egress from, the treatment area 28 when the inflatable wall is inflated. The main access opening 66 can be spaced apart from the top and bottom edges of the inflatable wall 24, and defined by respective access holes that extend through the inner and outer sidewalls 34, 36, and a main access sleeve 68 formed from fabric. The opposite annular ends of the main access sleeve 68 can be respectively connected securely to respective annular edges of the inner and outer sidewalls 34, 36 by sewn seams, so that the main access opening 66 extends through the inflatable wall 24 and is substantially isolated from the wall interior space 38.

The main access opening 66 can be selectively opened and closed. For example, the main chamber 22 can include an apparatus, or more specifically a door panel 70, configurable to at least partially close, or more specifically completely close, the main access opening 66. Referring to FIG. 4, the door panel 70 can include at least one layer of flexible foam insulation 72, or the like, securely sewn in place between/sewn to each of inner and outer fabric panels 74 of the door. The outer peripheral edge of the door 70 can have a permanently attached segment and a releasably attachable segment. Along the permanently attached segment, one or more of the fabric panels 74 of the door 70 can be connected to the main access sleeve 68 and/or closely associated edges of the inner and outer sidewalls 34, 36 by at least one elongate sewn seam. Referring to FIGS. 1 and 2, at least one releasable fastener or a portion thereof, such as an interlinkable strip 76 of a zipper 76, 78, can be securely connected to and extend along the releasable attachable segment of the door 70, for releasably mating with a corresponding interlinkable zipper strip 78 that is securely connected to and extends along a corresponding portion of the main access sleeve 68 and/or closely associated edges of the inner and/or outer sidewalls 34, 36. Accordingly, in one embodiment, the zipper 76, 78 and door 70 are configured for use in selectively opening and closing the main access opening 66.

There can be one or more openings from outside of the main chamber 22 to the wall interior space 38. Referring to FIG. 4, the outer sidewall 36 can include at least one inlet hole or opening 80 that is open to the wall interior space 38, for being in fluid communication with the inflation system 26 (FIG. 3). Alternatively, the inlet opening 80 can be located in another suitable location in the main chamber 22 or inflatable wall 24.

In the embodiment illustrated in the drawings, the inflation system 26 includes one or more modular devices that can be configured for being removably, modularly associated with the main chamber 22, such as for inflating the inflatable wall 24. For example, the inflation system 26 can include an electrically powered motorized blower 82 configured to be in fluid communication with the inlet opening 80 and, thus, the wall interior space 38, by way of at least one passageway that can be in the form of a fabric tube 84. Opposite ends of the tube 84 can be modularly, releasably connected to the discharge opening of the blower 82 and the inlet opening 80 by one or more releasable fasteners, such as hook and loop fastener strips, one or more straps, one or more zippers and/or any other suitable releasable fasteners. In one example, the blower 82 may have a half horsepower electric motor and be configured to provide a maximum flow of about six hundred cubic feet per minute, although any other suitable blower, or the like, may be used.

Referring to FIGS. 3 and 4, a secondary access opening 86 can extend through the inflatable wall 24 for having components of the cooling system 30 extend therethrough. The secondary access opening 86 can be spaced apart from the top and bottom edges of the inflatable wall 24, and defined by respective access holes that extend through the inner and outer sidewalls 34, 36, and a secondary access sleeve 88 formed from fabric. The opposite annular ends of the secondary access sleeve 88 can be respectively connected securely to respective annular edges of the inner and outer sidewalls 34, 36 by sewn seams, so that the secondary access opening 86 extends through the inflatable wall 24 and is substantially isolated from the wall interior space 38.

In the embodiment illustrated in the drawings, the cooling system 30 includes one or more modular devices that can be configured for being removably, modularly associated with the main chamber 22 for controllably providing vaporized cryogen to the treatment area 28. Referring to FIG. 3, the cooling system 30 can include least one passageway (e.g., comprising one or more hoses 90, 92) configured to supply liquid cryogen from a tank 94 or other suitable source into the treatment area 28, where the cryogen may be sprayed into the treatment area. More specifically and referring to FIG. 4, the cryogen can be sprayed into the interior of a modular diffuser assembly 96 that that can be removably mounted in the treatment area 28, wherein the interior of the modular diffuser assembly is open to the treatment area, as will be discussed in greater detail below.

As alluded to above, the at least one passageway for transporting the cryogen can include upstream and downstream flexible metal, cryogenic hoses 90, 92. The upstream hose 90 can be positioned between the tank 94 and a modular power and control unit 98. The downstream hose 92 can be positioned between the control unit 98 and the diffuser assembly 96 in the treatment area 28, so that the downstream hose 92 extends through the secondary access opening 86 (FIG. 4). The upstream end of the upstream hose 90 can include a fitting for being releasably connected to a conventional manually-operated discharge valve (not shown) of the tank 94, so that when the discharge valve is open liquid cryogen under pressure is supplied into the upstream hose.

Referring to FIG. 7, the downstream end of the upstream hose 90 can include a fitting for being releasably connected to an inlet fitting of a normally closed solenoid-operated valve 100 mounted in a housing of the control unit 98. The upstream end of the downstream hose 92 can include a fitting for being releasably connected to an outlet fitting of the solenoid valve 100, so that when the solenoid valve is open liquid cryogen under pressure can be supplied into the downstream hose. Referring to FIG. 4, the downstream hose 92 can extend through the second access opening 86.

Referring to FIGS. 8 and 9, the downstream end of the downstream hose 92 can include a fitting for being releasably connected to an inlet fitting 102 (FIG. 8) of a nozzle assembly 104. The nozzle assembly 104 can comprise at least one nozzle 107. More specifically, the nozzle assembly 104 can comprise or be in the form of a multi-port fitting 103 having a series of ports that are in fluid communication with the inlet fitting 102, wherein some of the ports can be respectively closed by plugs 105, and other of the ports can have nozzles 107 fixedly mounted therein for spraying the cryogen. The nozzles 107 can be configured for discharging in a manner that provides evaporative cooling, misting and/or fogging. As an example, suitable nozzles 107 may be MICROWHIRL® brand nozzles available from BETE Fog Nozzle, Inc. of Greenfield, Mass. and/or nozzles described by U.S. Pat. No. 7,198,201, although other suitable nozzle assemblies 104 and nozzles 107 may be used.

Referring to FIG. 9, the diffuser assembly 96 can include a rigid diffuser box 106 having front, top and rear partitions or wall boards 108 and spacers 120 respectively connected to one another so that right, left and bottom sides of the diffuser box are open to both the interior space 122 of the diffuser box. The nozzle assembly 104 can be mounted to the rear board 108 of the diffuser box 106 and extend into, or at least face, the box interior space 122 for discharging (e.g., spraying a mist of) the cryogen into the interior space of the diffuser box. For example, it is believed that the liquid cryogen mist sprayed from the nozzle assembly 104 into the box interior space 122 may substantially fully evaporate in the interior space of the diffuser box so that the resulting cold cryogenic gas flows outwardly through the open right, left and bottom sides of the diffuser box. Perhaps a relatively small amount of the unevaporated cryogenic mist may flow outwardly through the open right, left and bottom sides of the diffuser box 106.

For monitoring the temperature within the box interior space 122 and, thus, at least the approximate temperature in the treatment area 28, at least one temperature sensor 124 (e.g., resistance temperature detector (RTD)) can be mounted to the diffuser box 106 and extend into the box interior space 122, typically so that there is a substantial gap of at least several inches between the temperature sensor and the nozzle assembly 104. Alternatively, the nozzle assembly 104 and/or the temperature sensor 124 can be operatively associated with (e.g., positioned in, mounted in and/or mounted in sufficient proximity to) the treatment area 28 in any other suitable manner.

As depicted in FIG. 9, the diffuser assembly 96 can further include a box-shaped, fabric, flexible diffuser pouch or outer container 126 having front, right, left, rear, bottom and top fabric wall panels 128 for at least partially enclosing, or more specifically substantially fully enclosing, the diffuser box 106. The rear edge of the top wall panel 128 can be pivotably connected to the rear wall panel 128 so the top wall panel can be pivoted open to provide an access opening for receiving the diffuser box 106 therethrough. One or more fasteners can be provided for holding the top wall panel 128 in its closed configuration. For example, the flexible outer container 126 can further include one or more closure flaps 134 respectively pivotably connected to the front, right and left edges of the top wall panel 128. The closure flap(s) 134 can have fixedly mounted thereto releasable fastener(s), such as hook and loop fastener strips 138 for releasably connecting to corresponding hook and loop fastener strips 138 fixedly mounted to the front, right and left wall panels 128. The bottom, right and left wall panels 128 can comprise, consist essentially of, or consist of an open mesh material for allowing the cryogenic gas to flow outwardly from the box interior space 122 through the mesh material. As depicted in FIG. 9, the rear wall panel 128 can have a hole extending therethrough for having the nozzle fitting 102, downstream hose 92 and electrical wiring 140 extend therethrough.

Referring to FIGS. 10 and 11, the modular diffuser assembly 96 can be securely positioned in the treatment area 28 by being releasably mounted to the inner sidewall 34 using releasable fastener(s). For example, at least one mounting flap 142 pivotably connected to the rear edge of the top wall panel 128 and/or the top edge of the rear wall panel 128, or otherwise suitably configured, can have fixedly mounted thereto at least one hook and loop fastener strip 138 for releasably connecting to a corresponding hook and loop fastener strip 138 fixedly mounted to the inner sidewall 34. Similarly, a lower margin of a fabric diffuser panel 144 can be releasably mounted to the inner sidewall 34 by releasable fastener(s), and an upper margin of the diffuser panel can be releasably mounted by fastener(s) to the front wall panel 128 or another suitable portion of the outer container 126 or another suitable structure. These fastener(s) associated with the diffuser panel 144 can be hook and loop fastener strips 138 respectively fixedly mounted to the diffuser panel 144, inner sidewall 24 and front wall panel 128.

Referring to FIG. 7, the sensor electrical wiring 140, and similarly an electrical power cord 146, can be configured for electrically mating with corresponding fittings that can be mounted to the housing of the control unit 98. Buy way of a suitable electrical bus and/or electrical wiring schematically depicted in FIG. 7 as being within the housing of the control unit 98, the sensor electrical wiring 140 from the conventional temperature sensor 124 can be in electrical communication with a conventional signal converter 148, the conventional solenoid valve 100 can be in electrical communication with a conventional electrical relay 150, and the signal converter and electrical relay can be in electrical communication with respective inputs and outputs of at least one digital, electronic computer 152 (e.g., programmable logic controller and/or any other suitable computer device). The computer 152 can include or be operatively associated with at least one user interface 154 (FIGS. 3) that can be in the form of a touchscreen input device overlying an electronic visual display, for allowing a user or operator to easily operate at least some of the features of the cooling system 30 and/or other features of the cryotherapy system 20, as discussed in greater detail below. The user interface 154 can be mounted to the housing of the control unit 98 or be in any other suitable configuration. The user interface can more generally comprise one or more of a variety of types of user interfaces that are typically associated with electronic computers for allowing a user or operator to control at least the cooling system 30 of the cryotherapy system 20. The control unit 98 can be a “stand-alone” unit that is compact and easily transportable, although variations are within the scope of this disclosure.

Numerous components of the cryotherapy system 20 can be modular, for ease of assembly, ease of providing any replacement parts, and/or the like. An example of a method of modularly assembling some of the components of the cryotherapy system 20 is described in the following, in accordance with an embodiment of this disclosure. An operator (e.g., someone assisting a user of the system 20) can first connect the inflatable wall 24 to the motorized blower 84 by way of the tube 84 and associated fastener(s), or in any other suitable manner. Then, the blower 84 can be turned on to inflate the inflatable wall 24. When the blower 82 has inflated the inflatable wall and continues to supply air under pressure to the wall interior space 38 (e.g., while the main chamber 22 is in its erect configuration), the operator can carry the diffuser assembly 96, with the hose 92 and sensor electrical wiring 140 already connected thereto, into the treatment area 28 by way of the main access opening 66. The operator inserts the downstream hose 92 and sensor electrical wiring 140 outwardly through the secondary access opening 86. Then, the diffuser assembly 96 and diffuser panel 144 can be mounted in the treatment area 28 using respective fastener strips 138 and/or other suitable fasteners. As schematically depicted by arrows in FIG. 7, the hoses 90, 92, sensor electrical wiring 140 and power cord 146 can be attached to the respective fittings of the control unit 98. Similarly, the upstream end of the upstream hose 90 can be connected to the valve of the tank 94.

An example of a method of using the assembled cryotherapy system 20 is described in the following, in accordance with an embodiment of this disclosure. In accordance with this embodiment, the blower 82 continues to be operated during use of the cryotherapy system 20, so that the entire wall interior space 38 is continually supplied with ambient air under pressure and is maintained at a substantially constant air pressure that is above atmospheric pressure, causing the main chamber 22 stay erected (e.g., upright) during use. A user enters the treatment area 28 through main access opening 66 and securely closes the door 70 with the associated zipper 76, 78 and/or other suitable fastener(s), and stands inside treatment area 28. Referring to FIG. 2, if necessary or helpful, the user may stand on one or more modular platforms 155 placed on the floor of the treatment area 28 so that their head is positioned outside of, and above, the treatment area during use of the cryotherapy system 20. Each platform 155 can comprise a board enclosed in fabric.

The operator can open the valve on the tank 94, which can contain liquid nitrogen or another suitable cryogen, so that the cryogen flows through the upstream hose 92 to the normally closed solenoid valve 100. The control unit 98 can be controlled by the operator, by way of the user interface 154, so that opening and closing of the solenoid valve 100 is controlled and the cryogen flows through the downstream hose 92 and is discharged from the nozzle assembly 104 in a controlled manner to cause evaporative cooling. In one example, the control unit 98 can be configured to control opening and closing of the solenoid valve 100 and, thus, the spraying of the cryogen into the diffuser box 106, so that the temperature in the treatment area 28 is maintained at approximately a predetermined temperature for approximately a predetermined period of time. In one example, the control unit 98 can be configured to allow the operator to select, by way of the user interface 154, a predetermined temperature in a range of from zero to three hundred degrees below zero Fahrenheit (e.g., cryogenic temperatures), and a predetermined time from zero to three minutes, although other suitable ranges may be used. The control unit 98 can be configured to be responsive to feedback from the temperature sensor 124 in a manner that seeks to maintain the temperature within the treatment area 28 to within about five or ten degrees of, or any suitable variation from, the predetermined temperature for the entire predetermined time, or the like, although variations in temperature, time and methods of operation are within the scope of this disclosure.

The air within the wall interior space 38 can function as insulation that helps to control the temperature (e.g., the uniformity of the temperature) in the treatment area 28, and the optional flange 44 can substantially close the upper opening to the treatment area in a manner that also seeks to help control the temperature e.g., the uniformity of the temperature) in the treatment area. In addition, in the embodiment depicted in the drawings, after the inflatable wall 24 is fully inflated and while the blower 82 continues to supply air under pressure to the wall interior space 38, one or more flows of air are continually provided (e.g., by way of “leakage”) from the wall interior space 38 to the treatment area 28 for blending and/or mixing purposes, as will be discussed in greater detail below. When present, the outward flows of air from the interior space 38, such as to the treatment area 28, may be provided in any suitable manner, examples of which are described in the following.

In the above-described embodiments where the main chamber 22 is at least partially formed from fabric pieces, those fabric pieces may be in the form of fabric sheet(s), laminated fabric sheet(s) or coated fabric sheet(s) that are substantially fluid impermeable and capable of satisfactorily withstanding cryogenic temperatures, or the like, and the thread used to form the associated sewn seams may similarly be substantially fluid impermeable and capable of satisfactorily withstanding cryogenic temperatures. In one aspect of this disclosure, the main chamber 22 can consist essentially of fabric materials, such as lightweight heavy-duty inflatable fabrics. For example, suitable fabrics can include 1000 denier CORDURA® brand nylon fabric, and the thread can be heavy duty nylon thread, or the like; although there may be other suitable fabrics and threads, and alternatively the fabric, seams and/or thread may be replaced with other suitable features.

In the above-discussed embodiment(s) in which the main chamber 22 comprises both fabric and thread that is substantially fluid impermeable under normal operating conditions of the cryotherapy system 20, at least the sewn seams of the main chamber 22/inflatable wall 24 can be formed so that the sewn seams leak under operating conditions of the system. For example, FIG. 5 depicts the inflatable wall 24 in an inflated condition due to ambient air under pressure being supplied into the wall interior space 38 from the blower 82, and outward leakage of ambient air under pressure from the wall interior space through the inner and outer seams 52, 54 is schematically represented by respective arrows in FIG. 5. The leaking can occur along the entire length of the sewn seams 52, 54. Similarly, FIG. 4 depicts the inflatable wall 24 in an inflated condition due to ambient air under pressure being supplied into the wall interior space 38 from the blower 82, and outward leakage of ambient air under pressure from the wall interior space through the inner seams 52 into the treatment area 28 is schematically represented by respective arrows in FIG. 4. Air flow is schematically represented by arrows with solid leader lines in FIGS. 4 and 5, and flow or cryogen (e.g., cryogenic gas) is schematically represented by arrows with dashed leader lines in FIG. 4. Since some embodiments of this disclosure comprise leaky seams as discussed above, the word “substantially” may be associated at least some of the above-discussed features or attributes that are affected by the leakiness. For example, the wall interior space 24 may be described as being substantially sealed closed, since it may not be completely sealed closed due to outward leakage from the wall interior space 24 by one or more of (e.g., each of) the inner and outer seams 52, 54. Alternatively, the leaky sewn seams may be replaced with any other suitable seams of the inflatable wall 24.

It is believed that the leakage of air from the sewn seams (e.g., inner and outer seams 52, 54) of the inflatable wall 24 can promote advantageous mixing and improved uniformity of the cryogen and associated temperature within the treatment area 28 and/or advantageously inhibit formation of condensation on the main chamber 22/inflatable wall 24. The leakage of air from the sewn seams is believed to comprise leakage through very small gaps defined by the sewn seams. For enhancing, mimicking and/or replacing the effect of the leakage from the inner seams 52 (e.g., for discharging air under pressure from the wall interior space 38 into the treatment area 28, for mixing with the cryogen), there can be one or more other gaps or other suitable mixing openings 156 (FIG. 4) configured to supply air under pressure from the wall interior space 38 to the treatment area 28. Each mixing opening 156 can be defined by a slit or other suitable cut through the inner sidewall 34, and the opposite edges formed by the slit can be selectively partially or fully joined together by a releasable fastener that can be in the form of a zipper 158 (FIG. 11) configured to throttle the air flow through the mixing opening. The opposite interlinkable strips of the zipper 158 can be respectively sewn to the subject edges of the inner sidewall 34.

Referring to FIG. 12, the inflatable wall 24 can further include a relatively large outlet opening or vent opening 160 for selectively venting the wall interior space 38 to outside of the main chamber 22, such as when the cryotherapy system 20 not in use, the blower 82 is off, and it is desired to collapse the main chamber 22. The vent opening 160 can be positioned between (e.g., interrupt the sewn seam between) the lower edge of the outer sidewall 36 and the outer edge of the base panel 32, or the vent opening can be located in another suitable location in the main chamber 22 or inflatable wall 24. The vent opening 160 can be selectively opened and closed by a releasable fastener that can be in the form of a zipper 162, 164. The opposite interlinkable strips 162, 164 of the zipper 162, 164 can be respectively sewn to the respective edges of the outer sidewall 36 and the base panel 32. Alternatively, the vent opening 160 and its associated releasable fastener, or the like, can be in any other suitable configuration.

The above-discussed boards 108 of the diffuser box 106 and boards within the platforms 155 typically are boards, panels or other elements that are capable of satisfactorily withstanding cryogenic temperatures, such as by not becoming too brittle. For example, the boards can be high density, light weight foam capable of satisfactorily withstanding cryogenic temperatures. As a more specific example, suitable boards may be cut from Precision Board available from Coastal Enterprises, Inc. of Orange, Calif., although other suitable boards, panels and the like are within the scope of this disclosure.

Regarding the above-discussed hook and loop fasteners, as an example, they may be VELCRO® brand hook and loop fasteners, although any other suitable fasteners may be used. More generally regarding the above-discussed releasable fasteners, whereas several suitable examples have been identified, this disclosure is not limited to specific types of fasteners (e.g., any suitable fasteners may be used, and one or more of the above-discussed releasable fasteners may optionally be replaced with a suitable permanent fastener or mounting feature, if reasonably appropriate to do so).

As at least alluded to above, the cryogen can be liquid nitrogen. Although nitrogen may be preferred, this disclosure is not limited to the use of nitrogen; any other suitable cryogen can be used, such as, but not limited to, liquid helium, or the like, as will be understood by those of ordinary skill in the art.

In one aspect of this disclosure, the cryotherapy system 20 may be used, for example, to provide cryotherapy treatment for athletic recovery, pain management, and beauty applications.

In one aspect of this disclosure, the cryotherapy system 20 can be collapsible, compact, lightweight, portable, and can be set up relatively quickly in a wide variety of venues.

In the specification and/or figures, typical embodiments of the invention have been disclosed. The present invention is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

1. A cryotherapy system, comprising:
at least one inflatable chamber configured to extend at least partially around, and at least partially define, a treatment area configured to contain both vaporized cryogen and at least a portion of a user's body to be cryogenically treated; and
a cooling system configurable to be in fluid communication with the treatment area and provide vaporized cryogen to the treatment area.

2. The cryotherapy system according to claim 1, wherein:
the at least one inflatable chamber defines an access opening configured to provide ingress to and egress from the treatment area when the at least one inflatable chamber is inflated; and
the system further comprises an apparatus configurable to at least partially close the access opening.

3. The cryotherapy system according to claim 1, wherein the at least one inflatable chamber:
comprises fabric;
defines an interior space configured to contain air under pressure; and
comprises a plurality of sewn seams configured to discharge air from the interior space into the treatment area to mix the vaporized cryogen.

4. A method of using the cryotherapy system of claim 1, comprising:
inflating the at least one inflatable chamber; and
then operating the cooling system to provide vaporized cryogen to the treatment area while the at least one inflatable chamber is inflated.

5. A cryotherapy system, comprising:
a chamber configured to define a treatment area configured to contain both vaporized cryogen and at least a portion of a user's body to be cryogenically treated, the chamber comprising
at least one inflatable wall configured to extend at least partially around the treatment area at least when the at least one inflatable wall is inflated,
an access opening configured to provide ingress to and egress from the treatment area at least when the at least one inflatable wall is inflated, and
an apparatus configurable to at least partially close the access opening; and
a cooling system configurable to be in fluid communication with the treatment area and provide vaporized cryogen to the treatment area.

6. The cryotherapy system according to claim 5, wherein apparatus, which is configurable to at least partially close the access opening, comprises a panel.

7. The cryotherapy system according to claim 5, wherein the at least one inflatable wall:
faces and at least partially defines the treatment area;
defines an interior space configured to contain air under pressure; and
is configured to discharge air from the interior space into the treatment area to mix the vaporized cryogen.

8. The cryotherapy system according to claim 5, wherein the at least one inflatable wall comprises a plurality of subchambers extending at least partially around the treatment area.

9. The cryotherapy system according to claim 8, wherein the plurality of subchambers comprises a plurality of inflatable subchambers extending at least partially around the treatment area.

10. The cryotherapy system according to claim 5, wherein the at least one inflatable wall:
faces and at least partially defines the treatment area;
defines an interior space configured to contain air under pressure; and
comprises a plurality of sewn seams configured to discharge air from the interior space into the treatment area to mix the vaporized cryogen.

11. The cryotherapy system according to claim 10, wherein the at least one inflatable wall comprises:
a fabric inner sidewall facing and extending at least partially around the treatment area, the inner sidewall comprising a plurality of fabric pieces respectively connected to one another by the plurality of sewn seams; and
a fabric outer sidewall extending at least partially around the inner sidewall.

12. The cryotherapy system according to claim 5, wherein the cooling system comprises:
at least one nozzle configured to spray cryogen into the treatment area so that the cryogen evaporates in the treatment area; and
a control system configured to control when the cryogen is sprayed into the treatment area via the at least one nozzle to control temperature within the treatment area.

13. A method of using the cryotherapy system of claim 5, comprising:
inflating the at least one inflatable wall; and
then operating the cooling system to provide vaporized cryogen to the treatment area while the at least one inflatable wall is inflated.

14. A cryotherapy system, comprising:
an erectable apparatus configurable between an erected configuration and a collapsed configuration, wherein in the erected configuration, the erectable apparatus is configured to define a treatment area configured to contain both vaporized cryogen and at least a portion of a user's body to be cryogenically treated, and wherein the erectable apparatus comprises
at least one sidewall configured to extend at least partially around the treatment area when the erectable apparatus is in the erected configuration, and
a plurality of chambers configured to be inflated to at least partially support the at least one sidewall in the erected configuration; and
a cooling system configurable to be in fluid communication with the treatment area and provide vaporized cryogen to the treatment area.

15. The cryotherapy system according to claim 14, wherein:
the at least one sidewall comprises an inner sidewall;
the plurality of chambers defines an inflatable wall that includes the inner sidewall; and
the inflatable wall further includes an outer sidewall extending at least partially around the inner sidewall.

16. The cryotherapy system according to claim 14, wherein:
the at least one sidewall is an inner sidewall;
the erectable apparatus further comprises an outer sidewall;
a chamber of the plurality of cambers comprises a portion of the inner sidewall and a portion of the outer sidewall; and
the chamber defines an interior space positioned between the portion of the inner sidewall and the portion of the outer sidewall.

17. The cryotherapy system according to claim 16, wherein:
interior space is configured to contain air under pressure; and
the inner sidewall is configured to discharge air from the interior space into the treatment area to mix the vaporized cryogen.

18. The cryotherapy system according to claim 17, wherein the inner sidewall comprises a plurality of sewn seams configured to discharge air from the interior space into the treatment area to mix the vaporized cryogen.

19. The cryotherapy system according to claim 14, wherein the cooling system comprises:
a nozzle configured to spray cryogen into the treatment area so that the cryogen evaporates in the treatment area; and
a control system configured to control when the cryogen is sprayed into the treatment area via the at least one nozzle to control temperature within the treatment area.

20. A method of using the cryotherapy system of claim 14, comprising:
inflating the plurality of chambers; and
then operating the cooling system to provide vaporized cryogen to the treatment area while the plurality of chambers are inflated.
